(19) United States
(12) Patent Application Publication
(10) Pub. No.: US 2005/0020552 A1
(43) Pub. Date: Jan. 27, 2005

(54) Pharmaceutical composition and method for transdermal drug delivery

(76) Inventors: Chaim Aschkenasy, Yerucham (IL); Oren Chen, Beer Sheva (IL); Rami Boochnik, Yerucham (IL); Eilon Asculai, Lehavim (IL); Chalil Abu-Gnim, Beer Sheva (IL); Amira Zeevi, Omer (IL)

Correspondence Address: MARTIN MOYNIHAN;c/o ANTHONY CASTORINA, SUITE 207, 2001 JEFFERSON DAVIS HIGHWAY, ARLINGTON, VA, 22202, US

(21) Appl. No.: 10/891,487

(22) Filed: Jul. 15, 2004

Related U.S. Application Data
(60) Provisional application No. 60/487,278, filed on Jul. 16, 2003.
(60) Provisional application No. 60/487,248, filed on Jul. 16, 2003.
(60) Provisional application No. 60/487,277, filed on Jul. 16, 2003.
(60) Provisional application No. 60/581,458, filed on Jun. 22, 2004.

(51) Int. Cl. A61K 31/57

(52) U.S. Cl. 514/177

(57) ABSTRACT

A pharmaceutical composition for transdermal administration of a hormone (e.g., testosterone), which includes isostearic acid as a penetration enhancer, and methods utilizing same for treating medical conditions in which elevating a hormone serum level is beneficial are disclosed.

This application claims the benefit of priority from U.S. Provisional Patent Applications Nos. 60/487,278, 60/487,248, and 60/487,277, filed Jul. 16, 2003, and U.S. Provisional Patent Application No. 60/581,458, filed Jun. 22, 2004, all of which are incorporated by reference as if filly set forth herein.

### FIELD AND BACKGROUND OF THE INVENTION

The present invention relates to novel compositions for the transdermal administration of testosterone and/or other hormones and to methods utilizing these compositions.

Drugs are ideally administered in such a way as to enable an optimal concentration of active agent to be delivered to the intended target site. Conventional routes of administration include ingestion, injection, inhalation, and topical application.

Oral administration is the most prevalent method of administering pharmacological medicaments. The medicament is generally incorporated into a tablet, capsule, or a liquid base, and then swallowed. The oral administration modality is often preferred because of its convenience. In addition, oral administration is generally non-threatening, painless, and simple to accomplish for most patients.

Nevertheless, oral administration of drugs suffers from several disadvantages. One disadvantage is that pediatric and geriatric patients frequently have difficulty swallowing pills and other solid dosage forms, and such patients often refuse to cooperate in swallowing a liquid medication. In addition, for many medicaments, the act of swallowing the medicament often requires fluids and increases gastric volume and the likelihood of nausea and vomiting.

Furthermore, drugs with short half-lives require repeated daily dosing (2 to 4 times daily), which can lead to inadequate compliance. The short plasma half life of the drug and frequent dosing regimen may result in “peaks” and “valleys” in the plasma concentration profile, which increases the likelihood of adverse side effects associated with the peak concentration, as well as decreased therapeutic effectiveness towards the end of the dosing interval.

A further problem associated with oral administration is that the rate of absorption of the drug into the bloodstream after swallowing varies from patient to patient. The absorption of the drug is dependent upon the movement of the drug from the stomach to the small and large intestines and the effects of secretions from these organs and on the resulting pH within the stomach and intestines. Anxiety and stress can dramatically reduce these movements and secretions, prevent or reduce the final effects of the drug, and delay onset of the drug's effects.

An additional disadvantage associated with oral administration is the filet that there is normally a substantial delay between the time of oral administration and the time that the therapeutic effect of the drug begins. As mentioned above, the drug is must pass through the gastrointestinal system in order to enter the bloodstream, which typically takes forty-five minutes or longer.

An additional disadvantage of oral administration is that many drugs almost immediately experience metabolism or inactivation. The veins from the stomach and the small and large intestines pass directly through the liver. Thus, drugs entering the bloodstream must first pass though the liver before distribution into the general blood circulation. More than sixty percent of most drugs (and essentially one hundred percent of certain drugs) are removed from the patients bloodstream during this “first pass” through the liver, resulting in poor bioavailability.

Furthermore, additional stress is placed on the liver as it removes the excess drug from the bloodstream. This is particularly severe if the drug treatment has been occurring over an extended period of time. The liver may become overloaded with the drugs metabolite, which must then be excreted. As a result, there is an increased risk of hepatic or renal disorders.

Transdermal delivery of drugs provides many advantages over conventional oral administration. Advantages of transdermal systems include bypassing the portal circulation, thereby eliminating first-pass metabolism in the liver, convenience, non-interrupted therapy, improved patient compliance, reversibility of treatment (by removal of the transdermal system from the skin), and delivery of medication directly into the system circulation at a constant rate.

Although transdermal systems have many advantages over oral administration, most drugs are not amenable to this mode of administration due to the well-known barrier properties of the skin. Skin is a structurally complex, relatively thick membrane. Molecules moving from the environment into and through intact skin must first penetrate the stratum corneum and any material on its surface. The molecule must then penetrate the viable epidermis, the papillary dermis, and then the capillary walls into the blood stream or lymph channels.

The stratum corneum, the outer horny layer of the skin, is a complex structure composed of dead, keratinized, metabolically inactive cells, which are closely packed together, and consist of an amorphous matrix of mainly lipoid and non-fibrous protein within which keratin filaments are distributed. The cells of the stratum corneum generally contain 20% water, while the cells of the stratum germinativum, situated below the stratum corneum, contain 70% water. The high degree of keratinization within these cells, as well as their dense packing, creates a substantially impermeable barrier to drug penetration, presenting a rate-limiting barrier to absorption of topical compositions or transdermally administered drugs.

In order to improve the penetration of drugs into the skin, a variety of techniques and materials have been developed. These include iontophoresis and ultrasound to improve penetration of drugs into the skin, and the use of formulations containing penetration enhancing compounds, surfactants, lipids and other aliphatic compounds and liposomes.

Penetration enhancers are materials that have a direct effect on the permeability of the skin barrier. Chemical penetration enhancers are believed to operate mainly in the intercellular spaces of the stratum corneum. The exact mechanisms by which many chemical penetration enhancers function have not been clearly elucidated; it is almost certain that they will have multiple effects once absorbed into the stratum corneum. Effects that have been documented include an alteration of the solvent potential of the stratum corneum's biochemical environment, and a disordering of the intercellular lipid matrix following insertion of the enhancer into the bilayer store [“Percutaneous Penetration Enhancers”, E. W. Smith and H. I. Maibach, Eds., CRC Press, 1995]. Modern investigative techniques have shown that many enhancers may operate via a disruption of the ordered structure of the intercellular lipid regions of the stratum corneum. The insertion of the enhancer molecule between the parallel carbon chains of the fatty acids is believed to enhance the fluidity of this environment, thereby facilitating the diffusion of the coadministered drug [“Percutaneous Penetration Enhancers”, E. W. Smith and H. I. Maibach, Eds., CRC Press, 1995].

One method for transdermal delivery involves the use of a patch, which relies on diffusion of the drug through a membrane. A number of transdermal patch delivery systems are known, all of which include at least one adhesive layer for attaching the patch to the target site. These transdermal patch delivery systems suffer some disadvantages, attributed, for example, to the skin irritation caused by the adhesive layer and to their incompatibility for patients having excessively oily or tender skin, or hairy skin.

Topical formulations have also been developed for transdermal delivery, which are applied directly to the skin and release the active ingredient at a rate that is dependent upon the thermodynamic activity of the drug in the formulation. Topical formulations may be applied in the form of, for example, a gel, a cream, an ointment, a paste, a lotion, a milk, a suspension, an aerosol, a spray, a foam, a serum, a swab, a pledget, a pad or a patch.

A topical formulation should be easy to apply, without being too runny, greasy, or otherwise inconvenient to use by the patient. Hydrogels are macromolecular networks that absorb water and swell, but do not dissolve in water, due to the presence of both hydrophilic fictional groups that provide for water absorption, and crosslinked polymers that give rise to aqueous insolubility. Hydroalcoholic gels are hydrogels having a high alcohol concentration. These gels have the advantage of ease of administration. At the application site, the alcohol evaporates and it is believed that the drug becomes supersaturated. The skin functions as a reservoir for the drug, which is delivered to the systemic blood circulation at a relatively constant rate and during a period lasting several hours.

Testosterone is the primary endogenous male steroid hormone, produced primarily by the Leydig's cells in the testes in varying amounts throughout a person's lifespan.

The effects of this hormone become most evident during the time of puberty, when an increased output of testosterone will elicit dramatic physiological changes in the male body. This includes the onset of secondary male characteristics such as a deepened voice, body and facial hair growth, increased oil output by the sebaceous glands, development of sexual organs, maturation of sperm and an increased libido. Indeed the male reproductive system will not function properly if testosterone levels are not significant. All such effects are considered the masculinizing or “androgenic” properties of this hormone. Increased testosterone production will also cause growth promoting or “anabolic” changes in the body, including an enhanced rate of protein synthesis (leading to muscle accumulation).

Testosterone also has a number of secondary effects, which are of great importance for the stressability and performance characteristics of the human organism. These include the maintaining of an anabolic metabolic situation, the restoration of the performance of man following exhausting exercise and increasing is the psychophysiological stressability and stress resistance.

Over 90% of the testosterone in the blood is bound to protein and the biologically active component is free testosterone representing 4 to 8% of the total concentration in the blood. The testosterone concentration in the blood is subject to a physiological daily cycle (maximum concentration in the morning) a seasonal cycle (lowest concentration in May) and influences by living circumstances and ageing processes.

Testosterone pharmacological uses include hormone replacement therapy in males with a congenital or acquired deficiency or absence of endogenous testosterone (resulting in e.g. hypogonadism, erectile dysfunction), and treatment of AIDS wasting syndrome in HIV infected men.

Hypogonadism may be classified into one of three types. Primary hypogonadism includes testicular failure due to congenital or acquired anorchia, XYY Syndrome, XX males, Noonan's Syndrome, gonad dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica). Patients with primary hypogonadism show an intact feedback mechanism in that the low serum testosterone concentrations are associated with high follicle-stimulating hormone (FSH) and leutenizing hormone (LH) concentrations. However, because of testicular or other failures, the high LH concentrations are not effective at stimulating testosterone production. Secondary hypogonadism involve an idiopathic gonadotropin or LH-releasing hormone deficiency. This type of hypogonadism includes Kaliman's Syndrome, Prader-Labhart-Willi's Syndrome, Laurence-Moon-Biedl's Syndrome, pituitary insufficiency/adenomas, Pasqualim's Syndrome, hemochromatosis, hyperprolactinemia, or pituitary-hypothalamic injury from tumors, trauma, radiation, or obesity. Because patients with secondary hypogonadism do not demonstrate an intact feedback pathway, the lower testosterone concentrations are not associated with increased LH or FSH levels. Thus, these men have low testosterone serum levels but have gonadotropins in the normal to low range.

Third, hypogonadism may be age-related. Testosterone deficiencies in older is men may lead to a variety of physiological changes, including sexual dysfunction, decreased libido, loss of muscle mass, decreased bone density, depressed mood, and decreased cognitive function.

Symptoms of low testosterone include decreased sexual desire and ability (decreased libido), extreme tiredness, low energy, depression, and loss of certain male characteristics such as muscular build and deep voice.

The major physiological effects of androgens in normal women include, for example, anabolic effects on muscle, skin, hair and bone; stimulatory effects on erythropoiesis; modulatory effects on immune function; and psychological effects on mood, well-being and sexual function. In addition, endogenous androgens are important for the development of pubic hair and are thought to modulate the action of estrogens and progestins on a variety of reproductive target tissues. It is also believed that androgens play an important role in modulating the secretory function of the lacrimal gland.

Testosterone treatment is generally used in women to treat breast cancer and postpartum breast pain or engorgement, to enhance the sex drive, for relief of menopausal symptoms, restoration of lost energy, and to strengthen bone. Testosterone administration has also been found to be beneficial in young oophorectormized/hysterectomized women, post-menopausal women on estrogen replacement therapy, women on oral contrives, women with adrenal dysfunction, women with corticosteroid-induced adrenal suppression, and human immunodeficiency virus-positive women.

Testosterone is not effective when taken orally or by injection, because it is susceptible to relatively rapid breakdown by the liver. Systemic administration of testosterone should therefore preferably be effected transdermally.

While many efforts have been made to develop transdermal systems for the delivery of testosterone, in the form of a patch or a topical formulation applied directly to the skin of the subject, only a few have met with commercial success. These include, for example Androgel® and Testim™.

It is believed, in general, that many attempts to produce topical hormone replacement therapies have been unsuccessful due to the inability to adequately and stably target the systemic blood circulation with therapeutically effective dosages in a reasonable period of time. In addition, effective carrier systems, including, for example, solvents for the hormonal drug of interest and suitable percutaneous penetration enhancers, having the requisite product stability and drug delivery profiles, generally cannot be developed based simply on the knowledge of carrier systems in topical formulations for other specific drugs or even from the carriers for patch systems for the same drug.

The background art discloses various topical transdermal formulations for delivery of hormones and other active compounds, optionally using any of a wide range of penetration enhance. For example, U.S. Pat. No. 5,164,190; U.S. Pat. No. 5,152,997; U.S. Pat. No. 5,028,435; U.S. Pat. No. 5,288,498; EP 596903B1, and U.S. Pat. No. 4,704,282; US 2002/0014307 teaches use of a patch to deliver a vaporizable medicine, which may bc a hormone; U.S. Pat. No. 5,198,223, and U.S. Pat. No. 5,314,694 teach systems for transdermal administration of estrogens and progesterone; U.S. Pat. No. 4,788,062 teaches administration of progesterone and estradiol esters; U.S. Pat. No. 5,518,734 teaches administration of estradiol; U.S. Pat. No. 5,512,292 and U.S. Pat. No. 5,788,984 teach administration of estrogen and gestodene; U.S. Pat. No. 5,236,906 teaches a system for delivery of adrenocortical hormone and hyaluronic acid; U.S. Pat. No. 4,738,956 teaches delivery of hydrocortisone; U.S. Pat. No. 6,420,394 teaches delivery of non-steroidal anti-inflammatory drugs; U.S. Pat. No. 5,874,074 and U.S. Pat. No. 5,658,559 which teach a lotion comprising a dermatological agent, which may be a steroid; U.S. Pat. No. 5,904,931 teaches delivery of sex hormones; and U.S. Pat. No. 5,219,877 teaches delivery of an imidazole; US 2002/0111487 teaches transdermal administration of an active principle such as a hormone; US 2002/0099003 teaches a pharmaceutical composition which comprises a vasoactive agent, optionally together with a steroid such as testosterone; US 2003/109507 teaches delivery of progestin; US 2003/087885 teaches dihydrotestetone; and WO 02/22132 teaches delivery of progesterone.

US 2003/0022875 teaches an oral dosage form containing an androgenic agent, which may be coadministered together with a transdermal formulation including a vasoactive agent and a penetration enhancer.

Transdermal delivery systems in which testosterone is mentioned as one of a number of possible active agents include U.S. Pat. No. 5,505,958, U.S. Pat. No. 5,744,162; U.S. Pat. No. 5,891,463, U.S. Pat. No. 5,902,603, and US 2003/0082227, which teach use of various penetration enhancers in a system comprising a patch; U.S. Pat. No. 4,946,870, which teaches a system comprising a film-forming system comprising an aminopolysaccharide, and includes any one of a list of penetration enhancers; U.S. Pat. No. 6,267,984, which uses monoglyceride and ethyl palmitate as penetration enhancers; US 2002/0028235 which uses an ester sunscreen as penetration enhancer; U.S. Pat. No. 4,863,970, which uses a binary system of oleic acid, oleins, and oleyl alcohol with lower alcohols to enhance penetration; US 2002/0058650, which uses a penetration enhancing system comprising oleic acid, alcohol and glycol; US 2002/0150625, which teaches a poloxamer lecithin organogel as transdermal carrier; EP 644746, which teaches ethanol, water, glycerol monoleate and methyl laureate as enhancer; U.S. Pat. No. 6,019,988, which teaches use of separate permeation enhancer and drug compositions which are mixed at time of application; U.S. Pat. No. 6,562,369 and US 2003/0129220 which teach use of an inorganic base as penetration enhancer; and US 2003/091620, which teaches a quaternary ammonium salt penetration enhancer.

Use of various other penetration enhancers is taught in the background art. U.S. Pat. No. 4,804,541 teaches use of benzyl alcohol; WO 95/05137 teaches a permeation enhancer composition, which includes benzyl alcohol, propylene glycol monolaurate and a C2-C6 alkanediol; U.S. Pat. No. 5,760,096 teaches use of a glycol and an alcohol; U.S. Pat. No. 5,885,565 teaches use of a sterol; U.S. Pat. No. 6,319,913 teaches oleic acid; U.S. Pat. No. 5,723,114 teaches use of a proton pump inhibitor, U.S. Pat. No. 6,190,894 teaches enhancement of penetration by agents which cause inhibition of biosynthesis of epithelial components; U.S. Pat. No. 6,010,691 teaches stearylamine and transvaccenic acid; and U.S. Pat. No. 4,678,663 teaches a volatile silicone and a fatty alcohol. U.S. Pat. No. 5,894,019 teaches a system in which the active ingredient is dissolved in a liquid lipid to enhance penetration; and U.S. Pat. No. 5,023,252 teaches use of a compound which may be a macrocyclic ester, diester, amide, diamide, amidine, diamidine, thioester, dithioester, thioamide, ketone or lactone.

Topical transdermal formulation are also taught in the following scientific publications: Funke A P et al, Pharm Res 19:661-8 (2002); and Coldman M F et al., J Pharm Sci 58:1098-102 (1969).

In recent years, various papers and patents have been published, relating to use of hydroalcoholic gels for the transdermal delivery of hormones such as testosterone or dihydrotestosterone.

However, hydroalcoholic gels provide delivery rates which are generally not sufficiently high; therefore a large amount of gel must be applied to a relatively large skin surface area. Therefore, several publications and patents recommend the addition of certain penetration enhancers to the gel.

Examples of such publications include US 2003/0087885, which teaches delivery of dihydrotesterone, and U.S. Pat. No. 5,968,919 which teaches topical alcoholic or aqueous alcoholic gels containing testosterone, progesterone, or estradiol, with 2-n-nonyl-1,3-dioxolane or other hydrocarbyl derivative of 1,3-dioxolane or 1,3-1,3-dioxane or acetal as penetration enhancers

Another drawback of the hydroalcoholic gels is the sticky feeling caused by the gelling agents remaining on the skin after the evaporation of the alcohol. The commercial products Androgel® and Testim™ contain isopropyl myristate and pentadecalactone, respectively, as penetration enhancers. With both products a large amount of the product (5-10 grams) must be applied to the shoulders or the abdomen. With both products only a fraction of the applied testosterone reaches the systemic blood circulation.

The prior art therefore does not teach a topical composition for transdermal delivery of testosterone or related hormones, having a particularly effective penetration level and characterized by convenient and effective application.

Formulations having a penetration enhancer achieving desired serum drug levels cannot be developed based simply on the knowledge of carrier systems in topical formulations for other specific drugs. Moreover, even penetration enhancers belonging to the same chemical class (fatty acid and esters, polyols and surfactants among others) have sometimes quite different influences on penetration rates.

There is thus a widely recognized need for, and it would be highly advantageous to have, topical formulations, preferably hydrogel formulations, which include testosterone, and a highly effective penetration enhancer, and can therefore serve for transdermally delivering testosterone while being devoid of at least some of the above limitations.

### SUMMARY OF THE INVENTION

The present inventors have surprisingly found that topical compositions that comprise isostearic acid as a penetration enhancer are particularly effective systems for transdermal delivery of testosterone and related hormones.

Hence, according to one aspect of the present invention there is provided a pharmaceutical composition for topical application, which comprises a pharmaceutically active ingredient, a penetration enhancer, and a pharmaceutically acceptable carrier, wherein the pharmaceutically active ingredient is a hormone, and the penetration enhancer is isostearic acid.

The hormone is preferably any one or more of an androgenic hormone, an estrogenic hormone and a progestogenic hormone, and can be, for example, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrol, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone, estrone, estradiol and estriol, progesterone and pharmaceutically acceptable esters thereof, salts thereof, and combinations of any of the foregoing.

Preferably, the hormone is testosterone.

The concentration of the hormone preferably ranges between about 0.5 weight percentages and about 5 weight percentages, more preferably it is about 1 weight percentage.

In one embodiment of the present invention, the concentration of isostearic acid is lower than about 10 weight percentages, and more preferably it ranges between about 0.1 and about 4 weight percentages, more preferably between about 0.2 and about 2 weight percentages, more preferably between about 0.2 and about 1 weight percentages.

The composition can be formulated in a form of a gel, a cream, an ointment, a paste, a lotion, a milk, a suspension, an aerosol, a spray, a foam, a serum, a swab, a pledget, a pad and a patch. Preferably, the composition is formulated as a gel, and more preferably as a hydroalcoholic gel.

Such a hydroalcoholic gel pharmaceutical composition preferably comprises a C2-C4 alcohol, such as, for example, ethanol or isopropanol, preferably ethanol. The concentration of the C2-C4 alcohol preferably ranges between about 40 weight percentages and about 90 weight percentages, more preferably between about 55 weight percentages and about 70 weight percentages, and most preferably is about 69 weight percentages.

The hydroalcoholic gel composition preferably further comprises a gelling agent, such as, for example, a polymeric thickening agent, a fatty alcohol, a fatty acid, and a fatty acid alkali salt, an inorganic gelling agent and any mixture thereof, more preferably a polyacrylic acid or a cellulosic ether. Preferred cellulosic ethers are carboxymethylcellulose, hydroxypropyl cellulose and hydroxyethylcellulose. Preferred polymeric thickening at are xanthan gum and guar gum.

The concentration of the gelling agent preferably ranges between about 0.1 weight percentage and about 5 weight percentages, more preferably between about 0.1 weight percentage and about 2 weight percentages.

The pharmaceutical composition according to the present invention can further comprise a penetration co-enhancer, optionally and preferably a glycol.

The composition may optionally further comprise an additional pharmaceutically active ingredient.

The pharmaceutical composition optionally further comprises at least one additive, optionally and preferably selected from the group consisting of a moisturizing agent and an emollient. The additive optionally comprises glycerin. The additive may alternatively be an emollient selected from the group comprising dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof. Alternatively, the additive may be selected from the group consisting of a humectant, a deodorant agent, an antiperspirant, a pH adjusting agent, a stabilizing agent, a preservative, an emulsifier, an occlusive agent, a solubilizing agent, a colorant, and a surfactant. The concentration of additive preferably ranges between about 1 weight percentage and about 5 weight percentages.

In a preferred embodiment of the present invention, the pharmaceutical composition is a hydroalcoholic pharmaceutical composition, as described hereinabove, which comprises isostearic acid, testosterone, a C2-C4 alcohol and a gelling agent.

The pharmaceutical composition of the present invention is capable, upon application of an amount of the composition onto at least one biological surface of a subject, of elevating a blood serum concentration of the hormone in the subject from a subpotent concentration to a potent concentration within about 24 hours after application. For testosterone, in a human male, a potent concentration ranges from about 300 ng/dl to 1100 ng/dl in serum.

The amount of pharmaceutical composition preferably ranges between about 0.1 grams and about 10 grams. Alternatively, the amount of the composition preferably ranges between about 3 milligrams and 100 milligrams, more preferably between about 4 milligrams and about 60 milligrams per square centimeter of the biological surface In the case where the hormone is testosterone, the amount of composition may be lower than these values.

The biological surface can be, for example, the abdomen, an armpit, an inside arm, the back, a thigh, a shoulder, or the scrotum.

The pharmaceutical composition can therefore be packaged in a packaging material and identified in print, in or on the packaging material, for use in the treatment of a medical condition in which elevating a blood serum hormone level in a subject is beneficial.

In cases where the subject is a human male, the medical condition can be, for example, primary hypogonadism, secondary hypogonadism, age-related hypogonadism, hormone deficiency, erectile dysfunction, AIDS wasting syndrome, reduced sex drive, energy loss, loss of bone mass extreme tiredness, low energy, and/or depression.

In cases where the subject is a human female, the medical condition can be, for example, breast cancer, postpartum breast pain or engorgement, reduced sex drive, menopausal symptoms, energy loss, loss of bone mass, extreme tiredness, low energy, and/or depression. The human female may be, for example, young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

According to another aspect of the present invention there is provided a method of transdermally delivering a hormone to the blood serum of a subject. This method comprises providing a pharmaceutical composition for topical application which includes the hormone, isostearic acid, and a pharmaceutically acceptable carrier; and contacting an amount (e.g., about 5 grams for a composition comprising testosterone) of the topical pharmaceutical composition with at least one biological surface of the subject, to thereby deliver the hormone to the blood serum through the biological surface.

According to still another aspect of the present invention there is provided a method of treating a medical condition in which elevating a hormone serum level is beneficial. The method comprises topically applying onto one or more biological surface(s) of a subject in need thereof a pharmaceutically effective amount of the composition described hereinabove.

The hormone blood serum level, the amount of the composition, the subject, the medical condition and the biological surface are as described hereinabove.

An advantage of the compositions and/or the methods of the present invention is that the transdermal penetration of a hormone is substantially increased.

A further advantage of the compositions and/or the methods of the present invention is that a smaller amount of a hormone can be administered, while still achieving a desired serum hormone level.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the term “isostearic acid” refers to an isostearic acid per se or to a mixture of related compounds, the predominant ingredient of which is isostearic acid. This nomenclature is in accordance with the International Cosmetic Ingredient Dictionary and Handbook, Tenth Edition, 2004, vol. 1, published by the Cosmetic, Toiletry and Fragrance Association, which states that mixtures of similar materials are named on the basis of the predominant component.

As used herein, the phrase “topical application” describes application onto a biological surface. Hence, the phrase “a composition for topical application” describes a composition that is applied to a subject by contacting one or more biological surface(s) of the subject.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “pharmaceutically active ingredient” refers to a pharmaceutical agent including any natural or synthetic chemical substance that subsequent to its application has, at the very least, at least one desired pharmaceutical effect.

The term “penetration enhancement” refers to an increase in the permeability of skin to a pharmacologically active agent, so as to increase the rate at which the drug permeates though the skin and enters the bloodstream. The enhancement can be observed by mea the rate of diffusion of drug through animal or human skin using, for example a Franz diffusion apparatus as known in the art.

“Carriers” or “vehicles' refer to carrier materials suitable for transdermal drug administration and include any such material known in the art, e.g. any liquid, gel, solvent, liquid diluent, solubilizer or the like, which is nontoxic and which does not interact with other components of the composition in a deleterious manner. Examples of suitable carriers include water, alcohols, mineral oil, silicone, liquid sugars, waxes, petroleum jelly, and a variety of other oils and polymeric materials.

The term “transdermal” is intended to denote both transdermal (or “percutaneous”) and transmucosal administration, i.e., delivery by passage of drug through the skin or mucosal tissue. Hence the terms “transdermal” and “transmucosal” are used interchangeably unless specifically stated otherwise.

The term “therapeutically effective amount” or “pharmaceutically effective amount” denotes that dose of pharmaceutically active ingredient that will provide the pharmacological effect for which the active ingredient is indicated.

By “drug composition”, “drug/enhancer composition” or any similar terminology is meant a formulated composition containing the drug to be transdermally delivered in combination with such “carriers” or “vehicles”, penetration enhancers excipients, or any other additives.

As used herein, the phrase “pharmaceutically acceptable carrier” describes a carrier that does not cause Significant irritation to an organism and does not abrogate the biological activity and properties of the applied active ingredient.

As used herein, the phrase “potent concentration” with regard to a hormone denotes a concentration at which the hormone exerts a physiological effect. The phrase “subpotent concentration” denotes a concentration of the hormone which is below the potent concentration level.

As used herein the term “about” refers to ±10%.

The phrase “weight percentage(s)” or “percent” describes the weight percentage(s) of an ingredient of the total weight of a composition containing the ingredient.

The phrase greater than” as used herein with respect to a numerical indication (e.g., a concentration) encompasses any number (integral or fractional) that is greater than the indicated number.

The phrase “lower than” as used herein with respect to a numerical indication (e.g., a concentration) encompasses any number (integral or fractional) that is lower than the indicated number.

The phrase “ranging between” or “ranges between” as used herein with respect to a first numerical indication and a second numerical indication, and the phrase “ranging from” or “ranges from” a first numerical indication “to” a second numerical indication, are used interchangeably, and are meant to include the first and second numerical indications.

### DESCRIPTION OF THE PREFERRED EMBODIMENTS

The present invention is of a novel composition for transdermal delivery of a hormone (e.g., testosterone), using isostearic acid as penetration enhancer, which can be beneficially used in the treatment of medical conditions in which elevating the hormone serum level in a subject is beneficial, such as, but not limited to, hypogonadism, erectile dysfunction, hormone deficiency, depression, AIDS wasting syndrome, breast cancer, postpartum breast pain or engorgement, reduced sex drive, menopausal symptoms, energy loss, and loss of bone mass.

The principles and operation of the compositions, processes and methods according to the present invention may be better understood with reference to the Examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The prior alt teaches various formulations for transdermally delivering testosterone or other hormones. Most of these formulations are in the form of a patch, and therefore suffer major limitations, as is discussed in detail hereinabove. Alternative forms, such as, for example, emulsions, creams, aqueous solutions, oils, ointments, and pastes generally have the disadvantages of being runny, greasy, or otherwise inconvenient to use by the patient.

While conceiving the present invention, it was envisioned that isostearic acid can be efficiently used to enhance the skin permeation of testosterone and other hormones, and thus, that a composition for topical application that comprises a hormone and isostearic acid can be used for efficient transdermal delivery of the hormone. It was further envisioned that such a composition could be advantageously used when formulated as a hydroalcoholic gel.

The prior art does not teach or suggest a topical composition comprising testosterone or other hormone as the main active ingredient, with isostearic acid as penetration enhancer.

Prior art formulations for hormone delivery teach the use of various penetration enhancers. In some of these prior art disclosures, isostearic acid is included in a list of suitable penetration enhancers, without teaching isostearic acid as a particularly effective choice of compound for use as such a penetration enhancer. These include US 200/300,72792, which teaches a patch system for transdermal delivery of a drug, including androgenic hormones such as testosterone; U.S. Pat. No. 5,733,572 and U.S. Pat. No. 6,312,715, which teach a delivery system comprising gas filled microspheres; U.S. Pat. No. 6,019,997 and U.S. Pat. No. 5,908,619, which teach a hydroalcoholic formulation, preferably in the form of a lotion or patch, for delivery of a pharmaceutical agent, which may be a hormone; US 2003/0105030, which teaches delivery of an alpha reductase inhibitor, optionally together with testosterone.

Isostearic acid is also mentioned in the prior art as one of a large number of possible penetration enhancers in formulations comprising hydroalcoholic gels. U.S. Pat. No. 6,503,894, WO 02/17926, US 2603/0022877, US 2003/0027804, US 2003/0139384 and US 2003/0050292 describe a hydroalcoholic gel comprising an androgenic or anabolic steroid, which may be testosterone, and a functional derivative of a fatty acid, of which isostearic acid is one of many examples, as penetration enhancer; US 2002/0183296 and WO 02/17927 teach a hydroalcoholic gel comprising testosterone and a fatty acid derivative penetration enhancer.

Pharmaceutical Research (1989), 6(3), pp. 244-7, teaches isostearic acids as penetration enhancers for naloxone, an opiate antagonist. The use of isostearic acid as penetration enhancer for delivery of hormones is not taught. Furthermore, this paper describes isostearic acid at a concentration of 10 weight percentages to be only a moderate penetration enhancer, thereby teaching away from the use of isostearic acid as a penetration enhancer at concentrations of 10 weight percentages or lower.

While reducing the present invention to practice, it was indeed found that using isostearic acid as a penetration enhancer for increasing the skin permeability of testosterone, results in substantially enhanced testosterone permeability. As is shown in the Examples section that follows, such an enhanced skin permeability enables the use of a minimal amount of the applied composition and a minimal skin surface area onto which the composition is applied, while still achieving a desired testosterone level in a receiving medium (e.g., serum). The amount of the pharmaceutical composition of the present invention which is required to produce the desired effect therefore preferably ranges between about 0.1 grams and about 10 grams. Alternatively, the amount of the composition ranges between about 3 milligrams and 100 milligrams, preferably between about 4 milligrams and about 60 milligrams per square centimeter of a biological surface onto which it is applied.

Hence, according to one aspect of the present invention, there is provided a pharmaceutical composition for topical application, which comprises a hormone, as a pharmaceutically active ingredient, isostearic acid as a penetration enhancer, and a pharmaceutically acceptable carrier, and which is aimed at enhancing the skin penetrating effect of the active ingredient.

According to the present invention, the pharmaceutically active ingredient is a hormone. Suitable hormones for use in the context of the present invention include, for example, androgenic compounds, progestogenic compounds, including progestin compounds and progesterone, estrogenic compounds, and a combination thereof.

Representative example of androgenic compounds include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, pharmaceutically acceptable esters thereof, salts thereof, and combinations of any of the foregoing.

Representative examples of progestogenic compounds include, without limitation, progesterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-30-ol-20-one, 16,5α-pregnen-30-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone and combinations of any of the foregoing.

Representative examples of estrogen include, without limitation, estrone, estradiol, estriol and derivatives thereof.

In a preferred embodiment of the present invention, the hormone is an androgenic hormone, and, more preferably, it is testosterone.

The hormone concentration preferably ranges between about 0.5 weight percentage and about 5 weight percentages, more preferably between about 0.5 weight percentage and about 4 weight percentages, more preferably between about 0.5 weight percentage and about 3 weight percentages, more preferably between about 0.5 weight percentage and about 2 weight percentages, with a presently most preferred concentration being about 1 weight percentage.

As is shown in the Examples section that follows, it was surprisingly found that isostearic acid is an extremely effective penetration enhancer for delivery of a hormone. The concentration of isostearic acid needed to achieve an enhanced skin permeability of a hormone and as a result, an elevated level of the hormone in a receiving medium, as compared with a commercially available product, is relatively low.

Thus, a preferred concentration of isostearic acid is about 10 weight percentages, preferably lower than about 10 weight percentages of the total weight of the composition, more preferably lower than about 9 weight percentages, more preferably lower than about 8 weight percentages, more preferably lower than about 7 weight percentages, more preferably lower than about 6 weight percentages, more preferably lower than about 5 weight percentages, and even more preferably lower than about 4 weight percentages, such that a more preferred concentration of isostearic acid is about 3.5 weight percentages, more preferably about 3 weight percentages, more preferably about 2.5 weight percentages, more preferably about 2 weight percentages, more preferably about 1.5 weight percentages, and most preferably it is about 1 weight percentage of the total weight of the composition. The concentration of isostearic acid can be, however, even lower than 1 weight percentage and down to about 0.1 weight percentage.

Hence, the concentration of isostearic acid preferably ranges between about 0.1 and about 4 weight percentages, more preferably between about 0.1 and about 3 weight percentages, more preferably between about 0.1 and about 2 weight percentages, more preferably between about 0.2 and about 2 weight percentages, and more preferably between about 0.2 and about 1 weight percentages.

As is well known in the art, it is possible to enhance the effect of penetration enhancers by the co-inclusion of additional carriers or enhancers, such as glycols. Irritation caused by penetration enhancers or other ingredients may be reduced by using a combination of enhancers, thereby reducing the amount of each individual enhancer compound, or by the inclusion of non-irritating ingredients such as glycerin.

Hence, according to an embodiment of the present invention, the composition further comprises a penetration co-enhancer such as glycol or glycerin. Other suitable penetration co-enhancers for use in the context of the present invention include, without limitation, acetone, acyl lactylates, acyl peptides, acylsarcosinates, alkanolamine salts of fatty acids, alkyl benzene sulphonates, alkyl ether sulphates, alkyl sulphates, anionic surface-active agents, benzyl benzoate, benzyl salicylate, butan-1,4-ol, butyl benzoate, butyl laurate, butyl myrisite, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, ocetyl alcohol, octylphenoxy polyethoxyethanol, oleic ethanolamide, pleyl alcohol, pentan-2,4-iol, phenoxyethanol, phosphatidyl choline, phosphine oxides, polyalkoxylated ether glycollates, poly(diallylpiperidinium chloride), poly(dipropyldiallylammonium chloride), polyglycerol esters, polyoxyethylene lauryl ether, polyoxy:polyoxyethylene stearate, polyoxypropylene 15 stearyl ether, poly(vinyl pyridinium chloride), propan-1-ol, propan-2-ol, propylene glycol dipelargonate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sugar esters, sulphosuccinate, tetrahydrofuran tetrahydrofurfural alcohol, transcutol, triethanolamine dodecyl benzene sulphonate, triethanolamine oleate, water and derivatives, esters, salts and mixtures thereof.

The amount of enhancer used is selected so as to provide the desired delivery rate for the active ingredient, so as to enable application of a minimal amount of the composition onto a minimal skin surface area, taking into account the effect of additional factors such as product stability, side-effects, carrier system, and the like.

Further according to the present invention, the composition further comprises a pharmaceutically acceptable carrier.

Examples of pharmaceutically acceptable carriers that are usable in the context of the present invention include carrier materials that are well-known for use in the medical arts as bases for e.g., emulsions, creams, aqueous solutions, oils, ointments, pastes, gels, lotions, milks, foams, suspensions, aerosols, patches and the like, depending on the final form of the composition.

Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions.

Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

By selecting the appropriate carrier and optionally other ingredients that can be included in the composition, as is detailed hereinbelow, the composition of the present invention may be formulated into any form normally employed for topical application. Hence, the composition of the present invention can be, for example, in a form of a cream, an ointment, a paste, a gel, a lotion, a milk a suspension, an aerosol, a spray, a foam, a scrum, a swab, a pledget, a pad and a patch.

It will be appreciated that the final form of a topical composition plays an important role in its efficacy and its usage convenience. As is described above, gels, and particularly hydroalcoholic gels are highly advantageous for transdermal administration of drugs.

Hence, in a preferred embodiment of the present invention, the composition is formulated as a gel. More preferably, it is formulated as an aqueous gel, and more preferably as an aqueous-alcoholic gel (also referred to herein interchangeably as a hydroalcoholic gel). As is discussed hereinabove, a hydroalcoholic composition is highly advantageous in the context of the present invention.

Thus, in a preferred embodiment, the composition of the present invention further comprises an alcohol. Preferably, the alcohol is a C2-C4 alcohol such as, for example, ethanol and/or isopropanol. The concentration of the alcohol preferably ranges between about 40 weight percentages and about 90 weight percentages, more preferably between about 50 weight percentages and about 80 weight percentages, more preferably between about 55 weight percentages and about 75 weight percentages, more preferably between about 55 weight percentages and about 70 weight percentages and most preferably between about 65 weight percentages and about 70 weight percentages.

In another preferred embodiment, die composition of the present invention further comprises a gelling agent. Optionally and preferably, the gelling agent is a thickening agent, such as polyacrylic acid. However, any other pharmaceutically acceptable thickening/gelling agent may be used, such as hydroxypropyl cellulose, hydroxyethylcellulose, or other cellulosic ethers, other polymeric thickening agents such as xanthan gum, guar gum, and the like, fatty alcohols, fatty acids and their alkali salts and mixtures thereof, as well as inorganic thickeners/gelling agents.

The amount of gelling agent is not particularly critical, and can be selected to provide the desired product consistency or viscosity to allow for easy application to the skin. Generally, depending upon its molecular weight, amounts of thickening agent of up to about 5 weight percentages, preferably from about 0.1 weight percentages to about 2 weight percentages, of the composition will provide the desired effect.

The composition of the present invention may further comprise one or more inactive ingredients, which provide the compositions with additional usage benefits. Such inactive intents are referred to herein as “additives”. Examples of such additives include, but are not limited to, humectants, deodorant agents, antiperspirants, stabilizing agents, pH adjusting agents, preservatives, emulsifiers, occlusive agents, solubilizing agents, colorants, and surfactants.

Representative examples of pH adjusting agents that are usable in the context of the present invention include, without limitation, a base, an acid, a buffer system, or any combination thereof. Examples of such pH adjusting agents include, but are not limited to, bases such as ammonium hydroxide, sodium hydroxide, calcium hydroxide, trolamine, diisopropanolamine, and triisopropanolamine.

Representative examples of deodorant agents that are usable in the context of the present invention include, without limitation, quaternary ammonium compounds such as cetyl-trimethylammonium bromide, cetyl pyridinium chloride, benzethonium chloride, diisobutyl phonoxy ethoxy ethyl dimethyl benzyl ammonium chloride, sodium N-lauryl sarcosine, sodium N-palmithyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L lysine hexadecyl amide, heavy metal salt of citrate, salicylate, and piroctose, especially zinc salts, and acids thereof, heavy metal salts of pyrithione, especially zinc pyrithione and zinc phenolsulfate. Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the compositions of the present invention either in a solubilized or a particulate form and include, for example, aluminum or zirconium astringent salts or complexes.

Suitable preservatives that can be used in the context of the present composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof.

Suitable emulsifiers that can be used in the context of the present invention include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents that can be used in the context of the present invention include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Representative examples of solubilizing agents that are usable in this context of the present invention include, without limitation, complex-forming solubilizers such as citric acid, ethylenediaminetetraacetate, sodium meta-phosphate, succinic acid, urea, cyclodextrin, polyvinylpyrrolidone, diethylammonium-ortho-benzoate, and micelle-forming solubilizers such as tweens and spans e.g., TWEEN 80. Other solubilizers that are usable for the compositions of the present invention are, for example, polyoxyethylene sorbitan fatty acid ester, polyoxyethylene n-alkyl ethers, n-alkyl amine n-oxides, poloxamers, organic solvents, phospholipids and cyclodextrines.

In another preferred embodiment of the present invention, the composition includes at least one additional pharmaceutically active ingredient in addition to the hormone described above.

Suitable additional pharmaceutically active ingredients include but are not limited to active herbal extracts, acaricides, age spot and keratose removing agents, analgesics, local anesthetics, antiacne agents, antiaging agents, antibacterals, antibiotics, antiburn agents, antidepressants, antidermatitis agents, antiedemics, antihistamines, antihelminths, antihyperkeratolyte agents, antiinflammatory agents, antiirritants, antilipemics, antimicrobials, antimycotics, antioxidants, antipruritics, antipsoriatic agents, antirosacea agents, antiseborrheic agents, antiseptic, antiswelling agents, antiviral agents, antiyeast agents, astringents, topical cardiovascular agents, chemotherapeutic agents, corticosteroids, fungicides, hair growth regulators, hormones, hydroxyacids, insecticides, keratolytic agents, lactams, mitocides, non-steroidal anti-inflammatory agents, pediculicides, progestins, 5α reductase inhibitors, sanatives, scabicides, vasodilators and wart removers.

Suitable active herbal extracts include but are not limited to angelica, anise oil, astragali radix, azalea, benzyl acetate, birch tar oil, bornyl acetate, cacumen biotae, camphor, cantharidin, capsicum, cineole, cinnamon bark, cinnamon leaf, citronella, citronellol, citronellyl acetate, citronellyl formate, eucalyptus, eugenyl acetate, flos carthami, fructus mori, garlic, gerianiol, geranium, geranyl acetate, babanera, isobutyl angelicate, lavender, ledum latifolium, ledum palustre, lemongrass, limonene, linalool, linalyl acetate, methyl anthranilate, methyl cinnamate, mezereum, neem, nerol, neryl acetate, nettle root extract, oleum ricini, oregano, pinenes, α-pinene, β-pinene, radix angelicae sinesis, radix paenoiae rubra, radix polygoni multiflori, radix rehmanniae, rhizoma pinelliae, rhizoma zingiberis recens, sabadilla, sage, sandalwood oil, saw palmetto extract, semen sesami nigrum, staphysagria, tea tree oil, terpene alcohols, terpene hydrocarbons, terpene esters, terpinene, terpineol, terpinyl acetate and derivatives, esters, salts and mixtures thereof.

Suitable acaricides include but are not limited to amitraz, flumethrin, fluvalinate and derivatives, esters, salts and mixtures thereof.

Suitable age spot and keratoses removing agent include but are not limited to hydroxyacids, hydroquinone and derivatives, esters, salts and mixtures thereof.

Suitable analgesics include but are not limited to benzocaine, butamben picrate, dibucaine, dimethisoquin, dyclonine, lidocaine, pramoxine, tetracaine, salicylates and derivatives, esters, salts and mixtures thereof.

Suitable local anesthetics include but arm not limited to benzocaine, bupivacaine, butamben picrate, chlorprocaine, cocaine, dibucaine, dimethisoquin, dyclonine, etidocaine, hexylcaine, ketamine, lidocaine, mepivacaine, pramoxine, procaine, tetracaine, salicylates and derivatives, esters, salts and mixtures thereof.

Suitable antiacne agents include but are not limited to N-acetylcysteine, adapalene, azelaic acid,-benzoyl peroxide, cholate, clindamycin, deoxycholate, erythromycin, flavinoids, glycolic acid, meclocycline, metronidazole, mupirocin, octopirox, phenoxy ethanol, phenoxy proponol, pyruvic acid, resorcinol, retinoic acid, salicylic acid, seymnol sulfate, sulfacetamide-sulfur, sulfur, tazarotene, tetracycline, tretinoin triclosan and derivatives, esters, salts and mixtures thereof

Suitable antiaging agents, include but are not limited to melatonin and derivatives, esters, salts and mixtures thereof.

Suitable antibiotics include but are not limited to amanfadine hydrochloride, amanfadine sulfate, amikacin, amikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, famesol, floxacirlin, gentamicin, gentamicin sulfate, gramicidin, griseofulvin, haloprogin, haloquinol, hexachlorophene, iminocylcline, iodochlorhydroxyquin, kanamycin, kanamycin sulfate, lincomycin, lincomycin, lineomycin hydrochloride, macrolides, meclocycline, methacycline, methacycline hydrochloride, methenamine, methenamine hippurate, methenamine mandelate, methicillin, metronidazole, miconazole, miconazole hydrochloride, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin 0, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprimsulfamethoxazole, tylosin, vancomycin, yrothricin and derivatives, esters, salts and mixtures thereof.

Suitable antidepressants include but are not limited to norepinephrine-reuptake inhibitors, selective-serotonin-reuptake inhibitors, monoamine-oxidase inhibitors, serotonin-and-noradrenaline-reuptake inhibitors, corticotropin-releasing factor antagonists, αα-adrenoreceptor antagonists, NK1-receptor antagonists, 5-HT_{1A}-receptor agonist antagonists, amitriptyline, desmethylamitriptyline, clomipramine, doxepin, imipramine, imipramine-oxide, trimipramine, adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norcolipramine, noxiptilin, opipramol, perlapine, pizotyline, propizepine, quinupramine, reboxetine, tianeptine, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritansenn, sertraline, tandospirone, venlafaxinie and zimeldine and derivatives, esters, salts and mixtures thereof.

Suitable antihistamines include but are not limited to chlorcyclizine, diphenhydramine, mepyramine, methapyrilene, tripelennamine and derivatives, esters, salts and mixtures thereof.

Suitable antimycotics include but are not limited to azole compounds, butoconazole, chloroxine, ciclopirox olamine, clotrimazole, econazole, elubiol, fluconazole, griseofulvin, itraconazole, ketoconazole, mafenide acetate, miconazole, nystatin, oxiconazole, sulconazole, terbinafine, terconazole, tioconazole, undecylenic acid and derivatives, esters, salts and mixtures thereof.

Suitable antipruritics include but are not limited to menthol, methdilazine, trimeprazine, urea and derivatives, esters, salts and mixtures thereof.

Suitable antipsoriatic agents include but are not limited to 6-aminonicotinamide, 6-aminonicotinic acid, 2-aminopyrazinamide, anthralin, calcipotriene, 6-cabamoylinicotinamide, 6-chloronicotinamide, 2-carbamoylpyrazinamide, corticosteroids, 6-dimethylaminonicotinamide, dithranol, 6-formylaminonicotinamide, 6-hydroxy nicotinic, acid, 6-substituted nicotnamides, 6-substituted nicotinic acid, 2-substituted p namide, tazarotene, thionicotinamide, trichothcene mycotoxins and derivatives, esters, salts and mixtures thereof.

Suitable additional antirosacea agents include but are not limited to metronidazole, sulfacetamide and derivatives, esters, salts and mixtures thereof

Suitable antiseborrheic agents include but are not limited to glycolic acid, salicylic acid, selenium sulfide, zinc pyrithione and derivatives, esters, salts and mixtures thereof.

Suitable antiviral agents include but are not limited to acyclovir and derivatives, es, salts and mixtures thereof.

Suitable chemotherapeutic agents include but are not limited to daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.

Suitable corticosteroids include but are not limited to alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethsone valerate, budesonide, chloroprednisone, chloroprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, diflurosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, α-methyl dexamethasone, methylprednisolone, methylprednisolone acetate, mometasone furoate, paramethasone, prednisolone, prednisone, pregenolone, progesterone, spironolactone, triacinolone, triamcinolone acetonide and derivatives, esters, salts and mixtures thereof.

Suitable keratolytic agents include but are not limited to N-acetylcysteine, glycolic acid, pyruvic acid, resorcinol, sulfur, salicyclic add, retinoic acids and derivatives, esters, salts and mixtures thereof.

Suitable lactams include but are not limited to L-galactono-1,4-lactam, L-arabino-1,5-lactam, D-fucono-1,5-lactam, D-glucaro-1,4-lactam, D-glucurono-6,3-lactam, 2,5-tri-O-acetyl-D-glucurono-6,3-lactam, 2-acetamido-2-deoxyglucono-1,5-lactam, 2-acetamido-2-deoxygalactono-1,5-lactam, D-glucaro-1,4:6,3-dilactam, L-idaro-1,5-lactam, 2,3,5,tri-O-acetyl-D-glucaro-1,4-lactam, 2,5-di-O-acetyl-D-glucaro-1,4:6,3-dilactam, D-glucaro-1,5-lactam methyl ester, 2-propionoamide-2-deoxyglucaro-1,5-lactam and derivatives, esters, salts and mixtures thereof.

Suitable non-steroidal anti-inflammatory agent include but are not limited to oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamatcs, nefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, caprofen, oxaprozin, pranoprofen, miroprofen, tioxaprofen, suprofen, aminoprofen, tiaprofen, pyrazoles, phenylbutazone, oxyphenbutazone, feprazone, azapropazone, trimethmzone and derivatives, esters, salts and mixtures thereof.

Suitable pediculicides include but are not limited to DDT, lindane, malathion, permethrin and derivatives, esters, salts and mixtures thereof.

Suitable vasodilators include but are not limited to ethyl nicotinate, capsicum extract and derivatives, esters, salts and mixtures thereof.

Suitable wart removers include but are not limited to imiquimod, podophyllotoxin and derivatives, esters, salts and mixtures thereof.

The compositions of the present invention may be packed or presented in any convenient way. For example, they may be packed in a tube, a bottle, a unit dosage form or a pressurized container, using techniques well known to those skilled in the art and as set forth in reference works such as Remington's Pharmaceutical Science 15^{th} Ed. Optionally and preferably, the composition is packed in the form of a tube or a unit dosage form, such as a sachet. It is preferred that the packaging is done in such a way so as to minimize contact of the unused compositions with the environment, in order to minimize contamination of the compositions before and after the container is opened.

As is demonstrated in the Examples section that follows, the composition of the present invention can be efficiently used for transdermally administering testosterone. Such a transdermal delivery of testosterone or any other hormone evidently results in elevating the hormone serum level and can therefore be beneficial in the treatment of various conditions.

Thus, in a preferred embodiment of the present invention, the composition described hereinabove is packaged in a packaging material and is identified in print, in or on the package, for use in the treatment of a medical condition in which elevating a serum hormone level is beneficial. Examples or such medical conditions include, without limitation, hypogonadism, erectile dysfunction, hormone deficiency, AIDS wasting syndrome, breast cancer, postpartum breast pain or engorgement, reduced sex drive, depression, menopausal symptoms, energy loss, and loss of bone mass, as is detailed hereinbelow.

According to another aspect of the present invention, there is provided a method of transdermally delivering a hormone, such as testosterone, to the blood serum of a subject. The method is effected by providing an amount of a composition for topical application which comprises the hormone, isostearic acid, and a pharmaceutically acceptable carrier, as described hereinabove, and contacting an amount of the composition with one or more biological surface(s) of the subject, to thereby deliver the hormone to the blood serum of the subject through the biological surface(s).

The method according to this aspect of the present invention enables the use of a minimal amount of the applied composition and a minimal skin surface area onto which the composition is applied, while still achieving a desired hormone level in a receiving medium (e.g., serum), as is further detailed herein.

As can be seen by comparison of Examples 1 and 2, which compare the transdermal absorption through human skin of aqueous alcoholic gels comprising TWEEN-20, the commercially available product Androgel (containing isopropyl myristate, IPM, as a penetration enhancer) and aqueous alcoholic gels of the present invention, containing isostearic acid as a penetration enhancer, the amount of testosterone absorbed with isostearic acid as penetration enhancer was significantly higher than with either TWEEN-20 or isopropyl myristate. Specifically, the amount of testosterone in a receiver phase after 24 hours was found to be about 280 mcg with use of 1 weight percentage isostearic acid, and about 80 mcg with use of 0.7 weight percentages isopropyl myristate, while no difference was seen in formulations containing varying concentrations of Tween-20 as compared to those containing no penetration enhancer.

According to another aspect of the present invention, there is provided a method of treating a medical condition in which elevating a serum hormone level of a subject is beneficial. The method is effected by topically applying onto at least one biological surface of the subject, e.& an inside arm, the back, the abdomen, a thigh, an armpit, a shoulder, or the scrotum, a pharmaceutically effective amount of the composition of the present invention as described herein.

The method according to this aspect of the present invention may be used to treat a medical condition in a human male, which includes hormone replacement therapy in males with a congenital or acquired deficiency or absence of endogenous testosterone (resulting in e.g. hypogonadism, erectile dysfunction), and treatment of AIDS wasting syndrome in HIV infected men.

The hypogonadism may be primary hypogonadism such as testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica); secondary hypogonadism, including Kaliman's Syndrome, Prader-Labbart-Willi's Syndrome, Laurence-Moon-Biedl's Syndrome, pituitary insufficiency/adenomas, Pasqualim's Syndrome, hemochromatosis, hyperprolactinemia, or pituitary-hypothalamic injury from tumors, trauma, radiation, or obesity; or age-related hypogonadism, resulting in physiological changes, including sexual dysfunction, decreased libido, loss of muscle mass, decreased bone density, depressed mood, and decreased cognitive function.

Symptoms of low testosterone include decreased sexual desire and ability (decreased libido), extreme tiredness, low energy, depression, and loss of certain male characteristics such as muscular build and deep voice.

The method of the present invention may be used to treat a medical condition in a human female which includes breast cancer and postpartum breast pail or engorgement, to enhance the sex drive, for relief of menopausal symptoms, restoration of lost energy, and to strengthen bone. Testosterone administration has also been found to be beneficial in young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

According to this aspect of the present invention, the composition of the present invention may be co-administered together with an additional pharmaceutically active ingredient suitable for treating the medical condition. As a non-limiting example, the composition may be used in conjunction with pharmaceuticals aimed at improving sexual performance or impotence, including agents to treat erectile dysfunction, such as VIAGRA.RTM., or increasing libido by increasing testosterone levels in men. These pharmaceuticals can be administered orally, intravenously or via any other route of administration. In another example, the composition may be used in conjunction with antidepressants. In another example, non-drug therapies, such as, but not limited to, surgery, can be used in conjunction with the method according to this aspect of the present invention.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The phrase “topically applying” describes application onto one or more biological surface(s), by contacting a composition with the surface. Non-limiting examples of biological surfaces onto which the compositions of the present invention can be topically applied include one or more of the back, the abdomen, an inside arm, an armpit, a thigh, a shoulder, or the scrotum.

The composition is preferably applied to those regions of the skin which provide maximal systemic absorption of the hormonal active ingredient.

According to this aspect of the present invention, the composition of the present invention is preferably topically applied between two times a day and once in two days. More preferably, the composition is applied once a day, on a daily basis.

The phrase “pharmaceutically effective amount” describes an amount of a composition that is sufficient to significantly induce a positive modification in the condition being treated, but low enough to avoid significant side effects, within the scope of sound judgment of the skilled artisan. Preferably, the amount of the applied composition is sufficient to elevate the blood serum level of the administered hormone from a subpotent concentration to a potent concentration, within about 24 hours after the administration. In the case of testosterone in a human male subject, the potent blood serum concentration ranges from about 300 ng/dl to about 1100 ng/di.

A preferred amount of the composition of the present invention that upon application thereof results in the desired hormone level ranges between about 0.1 gram and 10 grams, preferably between about 1 gram and about 10 grams, more preferably between about 2 grams and about 10 grams, more preferably between about 3 grams and about 10 grains, more preferably between about 3 grams and about 10 grams, and more preferably between about 5 grams and about 10 grams. However, it should be noted that since due to enhanced penetration induced by isostearic acid, the amount of the composition of the present invention application thereof results in the desired hormone level can be less than 5 grams.

A representative example of a preferred composition according to an embodiment of the present invention, comprises about 1 weight percentage testosterone, and about 1 weight percentage isostearic acid, in a hydroalcoholic gel carrier, which enables the use of a minimal amount of the applied composition and a minimal skin surface area onto which the composition is applied, while sill achieving a desired testosterone level in a receiving medium (e.g., serum).

The present invention further encompasses processes for the preparation of the pharmaceutical compositions described above. These processes generally comprise admixing the active ingredients described hereinabove and the pharmaceutically acceptable carrier. In cases where other agents or active agents, as is detailed hereinabove, are present in the compositions, the process includes admixing these agents together with the active ingredients and the carrier. A variety of exemplary formulation techniques that are usable in the process of the present invention is described, for example, in Harry's Cosmeticology, Seventh Edition, Edited by J B Wilkinson and R J Moore, Longmann Scientific & Technical, 1982, Chapter 13 “The Manufacture of Cosmetics” pages 757-799.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

This example compares the transdermal absorption through human skin of testosterone from aqueous alcoholic gels containing 1.0% (w/w) testosterone, 0.0%, 0.1%, 0.7% or 2.0% of Tween-20 and 69.0% of ethanol. Carbopol 940 is used as the gelling agent in the gel formulations. The test compositions are applied to provide about 55 milligrams (mg) of the composition per square centimeter (cin) of human skin.

The tests are rum in standard diffusion cells with anol-water mixture (50:50) as the receptor fluid (surface area 1.77 cm², temperature 37 degree Celsius). The following Table 1 shows the concentration of the enhancer (Tween-20) in the formulations and the total amount of testosterone present in receiver phase after 24 hours for each formulation.

Each test was run for 24 hours under non-occluded conditions with the finite dose of the test formulation

TABLE 1

| | Total aamount of | Percentage of applied |
|---|---|---|
| Concentration of | testosterone in receiver | testosterone that reached |
| Tween 20 in form- | phase after 24 hours | receiver phase after 24 |
| ulation (% w/w) | (microgram) | hours (%) |
| 0.0 | 39.5 | 3.95 |
| 0.1 | 29.1 | 2.91 |
| 0.7 | 38.3 | 3.83 |
| 2.0 | 40.35 | 4.035 |

### Example 2

This example compares the transdermal absorption through human skin of testosterone from the following aqueous alcoholic gels containing about 1.0% (w/w) testosterone and about 69.0% ethanol: (1) Androgel (containing about 0.7% isopropyl myristate (IPM)); (2) a hydroalcoholic gel containing no added penetration enhancer, and (3) a hydroalcoholic gel containing 1.0% (w/w) isostearic acid. The isostearic used in these experiments is an isostearic acid mixture which comprises about 68% isostearic acid, together with various fatty acids. Carbopol 940 is used as the gelling agent in the gel formulations. The test compositions are applied to provide about 55 milligrams (mg) of the composition per square centimeter (cm²) of human skin.

The tests are run in standard diffusion cells with ethanol-water mixture (50:50) as the receptor fluid (surface area 1.77 cm²₇ temperature 37 degree Celsius). The following Table 2 shows the concentration of the enhancer (isosteric acid) in the formulations and the total amount of testosterone present in receiver phase after 24 hours for each formulation.

Each test was run for 24 hours under non-occluded conditions with the finite dose of the test formulation.

The results presented in Table 2 below clearly show that isostearic acid is a highly effective renetration enhancer, particularly as compared with isopropyl myristate, which is used as a penetration enhancer in a presently commercially available preparation for transdermal delivery of testosterone. As is shown in Table 1, the similar results obtained while using 0.7 wieght percentages of isopropyl myristae and 0.2 weight percentages isostearic acid, demonstrate the superior enhancement achieved with the latter.

TABLE 2

| | | Total amount of | Percentage of |
|---|---|---|---|
| | Concentration | testosterone in | applied testosterone |
| | of enhancer in | receiver phase after | that reached receiver |
| | formulation | 24 hours | phase after 24 hours |
| Enhancer | (% w/w) | (microgram) | (%) |
| No en- | No | 39.5 | 3.95 |
| hancer | | | |
| Isopropyl | 0.7 | 80.7 | 8.07 |
| myristate | | | |
| Isostearic | 0.2 | 78 | 7.8 |
| acid | | | |
| Isostearic | 0.5 | 183 | 18.3 |
| acid | | | |
| Isostearic | 1.0 | 280 | 28.0 |
| acid | | | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described with reference to specific embodiments thereof, many alternatives, modifications and variations will be apparent to those skilled in the art. Accordingly, it is intended that the present invention embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present Invention.

1. A pharmaceutical composition for topical application comprising a pharmaceutically active ingredient, a penetration enhancer and a pharmaceutically acceptable carrier, wherein said pharmaceutically active ingredient is a hormone, and said penetration enhancer is isostearic acid.

2. The pharmaceutical composition of claim 1, being capable, upon application of an amount of the composition onto at least one biological surface of a subject, of elevating a blood serum concentration of said hormone in said subject from a subpotent concentration to a potent concentration within about 24 hours after said application.

3. The pharmaceutical composition of claim 2, wherein said amount ranges between about 0.1 grams and about 10 grams.

4. The pharmaceutical composition of claim 2, wherein said amount ranges between about 3 milligrams and about 100 milligrams per square centimeter of said at least one biological surface.

5. The pharmaceutical composition of claim 4, wherein said amount ranges between about 4 milligrams and about 60 milligrams per square centimeter of said at least one biological surface.

6. The pharmaceutical composition of claim 1, wherein a concentration of said isostearic acid is equal to or lower than about 10 weight percentages.

7. The pharmaceutical composition of claim 6, wherein a concentration of said isostearic acid ranges between about 0.1 weight percentage and about 4 weight percentages.

8. The pharmaceutical composition of claim 7, wherein a concentration of said isostearic acid ranges between about 0.2 weight percentages and about 2 weight percentages.

9. The pharmaceutical composition of claim 8, wherein a concentration of said isostearic acid ranges between about 0.2 weight percentages and about 1 weight percentage.

10. The pharmaceutical composition of claim 1, wherein said hormone is selected from the group consisting of an androgenic hormone, an estrogenic; hormone and a progestogenic hormone.

11. The pharmaceutical composition of claim 10, wherein said hormone is selected from the group consisting of methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, desogestrel, dydrogesterone, ethynodiol diacetate, medroxyprogesterone, levonorgestrel, medroxyprogesterone acetate, hydroxyprogesterone caproate, norethidrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-2-one, 16,5α-pregnen-3β-ol-20-one, 4-pregnen-20β-ol-3-one-20-sulfate, acetoxypregnenolone, anagestone acetate, cyproterone, dihydrogesterone, flurogestone acetate, gestadene, hydroxyprogestrone acetate, hydroxymethylprogesterone, hydroxymethyl progesterone acetate, 3-ketodesogestrel, megestrol, melengestrol acetate, norethisterone, estrone, estradiol and estriol, progesterone, pharmaceutically acceptable esters thereof, salts thereof, and combinations of any of the foregoing.

12. The pharmaceutical composition of claim 11, wherein said hormone is testosterone.

13. The pharmaceutical composition of claim 1, wherein a concentration of said hormone ranges between about 0.5 weight percentages and about 5 weight percentages.

14. The pharmaceutical composition of claim 13, wherein a concentration of said hormone is about 1 weight percentage.

15. The pharmaceutical composition of claim 1, being formulated in a form selected from the group consisting of a gel, a cream, an ointment, a paste, a lotion, a milk, a suspension, an aerosol, a spray, a foam, a scrum, a swab, a pledget, a pad and a patch.

16. The pharmaceutical composition of claim 15, being formulated as a gel.

17. The pharmaceutical composition of claim 16, wherein said gel is a hydroalcoholic gel.

18. The pharmaceutical composition of claim 17, wherein said hydroalcoholic gel comprises a C2-C4 alcohol.

19. The pharmaceutical composition of claim 18, wherein said C2-C₄ alcohol is selected from the group comprising ethanol and isopropanol.

20. The pharmaceutical composition of claim 19, wherein said C2-C₄ alcohol is ethanol.

21. The pharmaceutical composition of claim 18, wherein a concentration of said C2-C4 alcohol ranges between about 40 weight percentages and about 90 weight percentages.

22. The pharmaceutical composition of claim 21, wherein a concentration of said C2-C4 alcohol ranges between about 55 weight percentages and about 70 weight percentages.

23. The pharmaceutical composition of claim 22, wherein a concentration of said C2-C4 alcohol is about 69 weight percentages.

24. The pharmaceutical composition of claim 16, further comprising a gelling agent.

25. The pharmaceutical composition of claim 24, wherein said gelling agent is selected from the group consisting of a polymeric thickening agent, a fatty alcohol, a fatty acid, and a fatty acid alkali salt, an inorganic gelling agent and any mixture thereof.

26. The pharmaceutical composition of claim 24, wherein said gelling agent comprises a polyacrylic acid.

27. The pharmaceutical composition of claim 25, wherein said polymeric thickening agent comprises a cellulosic ether.

28. The pharmaceutical composition of claim 27, wherein said cellulosic ether is selected from the group consisting of carboxymethylcellulose, hydroxypropyl cellulose and hydroxyethylcellulose.

29. The pharmaceutical composition of claim 25, wherein said polymeric thickening agent is selected from the group consisting of xanthan gum and guar gum.

30. The pharmaceutical composition of claim 24, wherein a concentration of said gelling agent images between about 0.1 weight percentage and about 5 weight percentages.

31. The pharmaceutical composition of claim 30, wherein a concentration of said gelling agent ranges between about 0.1 weight percentage and about 2 weight percentages.

32. The pharmaceutical composition of claim 1, farther comprising a penetration co-enhancer.

33. The pharmaceutical composition of claim 32, wherein said penetration co-enhancer is a glycol.

34. The pharmaceutical composition of claim 1, further comprising an additional pharmaceutically active ingredient.

35. The pharmaceutical composition of claim 1, further comprising at least one additive.

36. The pharmaceutical composition of claim 35, wherein said at least one additive is selected from the group consisting of a moisturizing agent and an emollient.

37. The pharmaceutical composition of claim 35, wherein a concentration of said at least one additive ranges between about 1 weight percentage and about 5 weight percentages.

38. The pharmaceutical composition of claim 35, wherein said at least one additive comprises glycerin.

39. The pharmaceutical composition of claim 36, wherein said emollient is selected from the group comprising dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

40. The pharmaceutical composition of claim 35, wherein said at least one additive is selected from the group consisting of a humectant, a deodorant agent, an antiperspirant, a stabilizing agent, a pH adjusting agent, a preservative, an emulsifier, an occlusive agent, a solubilizing agent, a colorant, and a surfactant.

41. The pharmaceutical composition of claim 1, packaged in a packaging material and identified in print, in or on said packaging material, for use in the treatment of a medical condition in which elevating a serum hormone level in a subject is beneficial.

42. The pharmaceutical composition of claim 41, wherein said subject is a human male.

43. The pharmaceutical composition of claim 42, wherein said medical condition is selected from the group consisting of primary hypogonadism, secondary hypogonadism, age-related hypogonadism, hormone deficiency, erectile dysfunction, AIDS wasting syndrome, reduced sex drive, energy loss, loss of bone mass, extreme tiredness, low energy, and depression.

44. The pharmaceutical composition of claim 41, wherein said subject is a human female.

45. The pharmaceutical composition of claim 44, wherein said medical condition is selected from the group consisting of breast cancer, postpartum breast pain or engorgement, reduced sex drive, menopausal symptoms, energy loss, loss of bone mass, extreme tiredness, low energy, and depression.

46. The pharmaceutical composition of claim 44, wherein said human female is selected from the group consisting of young oophorectomized/hysterectomized women, post-menopausal women on estrogen replacement therapy, women on oral contraceptives, women with adrenal dysfunction, women with cortcosteroid-induced adrenal suppression, and human immunodeficiency virus-positive women.

47. A hydroalcoholic pharmaceutical composition for topical application comprising testosterone, isostearic acid, a C2-C4 alcohol and a gelling agent.

48. The hydroalcoholic pharmaceutical composition of claim 47, being capable, upon application of an amount of the composition onto at least one biological surface of a subject, of elevating a blood serum concentration of said testosterone in said subject to a concentration that ranges between about 300 ng/dl and about 1100 ng/dl within about 24 hours after said application.

49. The hydroalcoholic pharmaceutical composition of claim 48, wherein said amount ranges between about 0.1 gams and about 10 grams.

50. The hydroalcoholic pharmaceutical composition of claim 47, wherein a concentration of said isostearic acid is equal to or lower than about 10 weight percentages.

51. The hydroalcoholic pharmaceutical composition of claim 50, wherein a concentration of said isostearic acid ranges between about 0.1 weight percentage and about 4 weight percentages.

52. The hydroalcoholic pharmaceutical composition of claim 51, wherein a concentration of said isostearic acid ranges between about 0.2 weight percentages and about 2 weight percentages.

53. The hydroalcoholic pharmaceutical composition of claim 52, wherein a concentration of said isostearic acid ranges between about 0.2 weight percentages and about 1 weight percentage.

54. The hydroalcoholic pharmaceutical composition of claim 47, wherein a concentration of said testosterone ranges between about 0.5 and about 5 weight percentages.

55. The hydroalcoholic pharmaceutical composition of claim 54, wherein a concentration of said testosterone is about 1 weight percentage.

56. The hydroalcoholic pharmaceutical composition of claim 47, Her comprising an additional pharmaceutically active ingredient.

57. The hydroalcoholic pharmaceutical composition of claim 47, packaged in a packaging material and identified in print, in or on said packaging material, for use in the treatment of a medical condition in which elevating a scrum hormone level in a subject is beneficial.

58. The hydroalcoholic pharmaceutical composition of claim 57, wherein said subject is a human male.

59. The hydroalcoholic pharmaceutical composition of claim 58, being capable, upon application of an amount of the composition onto at least one biological surface of said male subject, of elevating a blood serum concentration of said testosterone in said human male to a value ranging between about 300 ng/dl and about 1100 ng/dl within about 24 hours after said application.

60. The hydroalcoholic pharmaceutical composition of claim 58, wherein said medical condition is selected from the group consisting of primary hypogonadism, secondary hypogonadism, age-related hypogonadism, hormone deficiency, erectile dysfunction, AIDS wasting syndrome, reduced sex drive, energy loss, loss of bone mass, extreme tiredness, low energy, and depression.

61. The hydroalcoholic pharmaceutical composition of claim 57, wherein said subject is a human female.

62. The hydroalcoholic pharmaceutical composition of claim 61, wherein said medical condition is selected from the group consisting of breast cancer, postpartum breast pain or engorgement, reduced sex drive, menopausal symptoms, energy loss, loss of bone muss, extreme tiredness, low energy, and depression.

63. The hydroalcoholic pharmaceutical composition of claim 61, wherein said human female is selected from the group consisting of young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

64. A method of transdermally delivering a hormone to the blood serum of a subject, the method comprising:
providing a pharmaceutical composition for topical application including said hormone, isostearic acid, and a pharmaceutically acceptable carrier; and
contacting an amount of said topical pharmaceutical composition with at least one biological surface of said subject, to thereby deliver said hormone to said blood serum through said biological surface.

65. The method of claim 64, wherein said amount of said pharmaceutical composition ranges between about 0.1 gram and about 10 grams.

66. The method of claim 64, wherein said amount of said pharmaceutical composition ranges between about 3 milligrams and about 100 milligrams per square centimeter of said at least one biological suffice.

67. The method of claim 66, wherein said amount ranges between about 4 milligrams and about 60 milligrams per square centimeter of said at least one biological surface.

68. The method of claim 64, wherein a concentration of said hormone in said blood serum of said subject is elevated from a subpotent concentration to a potent concentration within about 24 hours after said contacting.

69. The method of claim 64, wherein said at least one biological surface is selected from the group consisting of the abdomen, an armpit, an inside arm, the back, a thigh, a shoulder, and the scrotum.

70. The method of claim 64, wherein a concentration of said isostearic acid is equal to or lower than about 10 weight percentages of the total weight of said composition.

71. The hydroalcoholic pharmaceutical composition of claim 70, wherein a concentration of said isostearic acid ranges between about 0.1 weight percentages and about 4 weight percentages.

72. The hydroalcoholic pharmaceutical composition of claim 71, wherein a concentration of said isostearic acid ranges between about 0.2 weight percentages and about 2 weight percentages.

73. The hydroalcoholic pharmaceutical composition of claim 72, wherein a concentration of said isostearic acid ranges between about 0.2 weight percentages and about 1 weight percentage.

74. The method of claim 64, wherein said hormone is selected from the group consisting of an androgenic hormone an estrogenic hormone and a progestogenic hormone.

75. The method of claim 74, wherein said hormone is selected from the group consisting of methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenodione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethrodrel, allylestrenol, 19-nortestosterone, lynoestrenol, quingestanol acetate, medrogestone, norgestrienone, dimethisterone, ethisterone, cyproterone acetate, chlormadinone acetate, megestol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-30-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestol acetate, norethisterone, estrone, estradiol and estriol, progesterone, pharmaceutically acceptable esters, salts thereof, and combinations of any of the foregoing.

76. The method of claim 75, wherein said hormone is testosterone.

77. The method of claim 64, wherein a concentration of said hormone ranges between about 0.5 weight percentages and about 5 weight percentages of the total weight of said composition.

78. The method of claim 77, wherein a concentration of said hormone is about 1 weight percentage of the total weight of said composition.

79. The method of claim 64, wherein said pharmaceutical composition is formulated in a form selected from the group consisting of a gel, a cream, an ointment, a paste, a lotion, a milk, a suspension, an aerosol, a spray, a foam, a serum, a swab, a pledget, a pad and a patch.

80. The method of claim 79, wherein said pharmaceutical composition is formulated as a gel.

81. The method of claim 80, wherein said gel is a hydroalcoholic gel.

82. The method of claim 81, wherein said hydroalcoholic gel comprises a C2-C4, alcohol.

83. The method of claim 82, wherein said C2-C4 alcohol is selected from the group comprising ethanol and isopropanol.

84. The method of claim 83, wherein said C2-C4 alcohol is ethanol.

85. The method of claim 82, wherein a concentration of said C2-C₄ alcohol ranges between about 40 weight percentages and about 90 weight percentages of the total weight of said composition.

86. The method of claim 85, wherein a concentration of said C2-C₄ alcohol ranges between about 55 weight percentages and about 70 weight percentages of the total weight of said composition.

87. The method of claim 86, wherein a concentration of said C2-C₄ alcohol is about 69 weight percentages of the total weight of said composition.

88. The method of claim 80, wherein said pharmaceutical composition further comprises a gelling agent.

89. The method of claim 88, wherein said gelling agent is selected from the group consisting of a polymeric thickening agent, a fatty alcohol, a fatty acid, and a fatty acid alkali salt, an inorganic gelling agent and any mixture thereof.

90. The method of claim 88, wherein said gelling agent comprises a polyacrylic acid.

91. The method of claim 89, wherein said polymeric thickening agent comprises a cellulosic ether.

92. The method of claim 91, wherein said cellulosic ether is selected from the group consisting of carboxymethylcellulose, hydroxypropyl cellulose and hydroxyethylcellulose.

93. The method of claim 89, wherein said polymeric thickening agent is selected from the group consisting of xanthan gum and guar gum.

94. The method of claim 88, wherein a concentration of said gelling agent ranges between about 0.1 weight percentage and about 5 weight percentages of the total weight of said composition.

95. The method of claim 94, wherein a concentration of said gelling agent ranges between about 0.1 weight percentage and about 2 weight percentages of the total weight of said composition.

96. The method of claim 64, wherein said pharmaceutical composition further comprises a penetration co-enhancer.

97. The method of claim 96, wherein said penetration co-enhancer is a glycol.

98. The method of claim 64, wherein said pharmaceutical composition further comprises an additional pharmaceutically active ingredient.

99. The method of claim 64, wherein said pharmaceutical composition further comprises at least one additive.

100. The method of claim 99, wherein said at least one additive is selected from the group consisting of a moisturizing agent and an emollient.

101. The method of claim 99, wherein a concentration of said at least one additive ranges between about 1 weight percentage and about 5 weight percentages of the total weight of said composition.

102. The method of claim 99, wherein said at least one additive comprises glycerin.

103. The method of claim 100, wherein said emollient is selected from the group comprising dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

104. The method of claim 99, wherein said at least one additive is selected from the group consisting of a humectant, a deodorant agent, an antiperspirant, a stabilizing agent, a pH adjusting agent, a preservative, an emulsifier, an occlusive agent, a solubilizing agent, a colorant, and a surfactant.

105. A method of treating a medical condition in which elevating a blood serum hormone level in a subject is beneficial, the method comprising:
providing a pharmaceutical composition for topical application including said hormone, isostearic acid and a pharmaceutically acceptable carrier;
topically applying onto at least one biological surface of said subject a pharmaceutically effective amount of said topical pharmaceutical composition, thereby elevating said blood serum hormone level in said subject and treating said medical condition.

106. The method of claim 105, wherein said pharmaceutically effective amount of said pharmaceutical composition ranges between about 0.1 gram and about 10 grams.

107. The method of claim 105, wherein said amount of said pharmaceutical composition ranges between about 3 milligrams and about 100 milligrams per square centimeter of said at least one biological surface.

108. The method of claim 107, wherein said amount of said pharmaceutical composition ranges between about 4 milligrams and about 60 milligrams per square centimeter of said at least one biological surface.

109. The method of claim 105, wherein said hormone level is elevated from a subpotent concentration to a potent concentration within about 24 hours after said topical application.

110. The method of claim 105, wherein said at least one biological surface is selected from the group consisting of the abdomen, an armpit, an inside arm, the back, a thigh, a shoulder, and the scrotum.

111. The method of claim 105, wherein a concentration of said isostearic acid is equal to or lower than about 10 weight percentages of the total weight of said composition.

112. The method of claim 111, wherein a concentration of said isostearic acid ranges between about 0.1 weight percentage and about 4 weight percentages of the total weight of said composition.

113. The method of claim 112, wherein a concentration of said isostearic acid ranges between about 0.2 weight percentage and about 2 weight percentages of the total weight of said composition.

114. The method of claim 113, wherein a concentration of said isostearic acid ranges between about 0.2 weight percentage and about 1 weight percentage of the total weight of said composition.

115. The method of claim 15, wherein said hormone is selected from the group consisting of an androgenic hormone, an estrogenic hormone and a progestogenic hormone.

116. The method of claim 115, wherein said hormone is selected from the group consisting of methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-iol sulfate, 5α-pregnan-30-ol-20-one, 16,5α-pregnen-3>ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone, estrone, estradiol and estriol, progesterone, pharmaceutically acceptable esters thereof, salts thereof, and combinations of any of the foregoing.

117. The method of claim 116, wherein said hormone is testosterone.

118. The method of claim 105, wherein a concentration of said hormone ranges between about 0.5 weight percentages and about 5 weight percentages of the total weigh of the said composition.

119. The method of claim 118, wherein a concentration of said hormone is about 1 weight percentage of the total weight of the said composition.

120. The method of claim 105, wherein said pharmaceutical composition is formulated in a form selected from the group consisting of a gel, a cream, an ointment, a paste, a lotion, a milk, a suspension, an aerosol, a % pray, a foam, a serum, a swab, a pledget a pad and a patch.

121. The method of claim 120, wherein said pharmaceutical composition is formulated as a gel.

122. The method of claim 121, wherein said gel is a hydroalcoholic gel.

123. The method of claim 122, wherein said hydroalcoholic gel comprises a C2-C4 alcohol.

124. The method of claim 123, wherein said C2-C4 alcohol is selected from the group comprising ethanol and isopropanol.

125. The method of claim 124, wherein said C2-C4 alcohol is ethanol.

126. The method of claim 123, wherein a concentration of said C2-C₄ alcohol ranges between about 40 weight percentages and about 90 weight percentages of the total weight of the said composition.

127. The method of claim 126, wherein a concentration of said C2-C₄ alcohol ranges between about 55 weight percentages and about 70 weight percentages of the total weight of the said composition.

128. The method of claim 127, wherein a concentration of said C2-C₄ alcohol is about 69 weight percentages of the total weight of the said composition.

129. The method of claim 121, wherein said pharmaceutical composition further comprises a gelling agent.

130. The method of claim 129, wherein said gelling agent is selected from the group consisting of a polymeric thickening agent a fatty alcohol, a fatty acid, and a fatty acid alkali salt, an inorganic gelling agent and any mixture thereof.

131. The method of claim 130, wherein said gelling agent comprises a polyacrylic acid.

132. The method of claim 130, wherein said polymeric thickening agent comprises a cellulosic ether.

133. The method of claim 132, wherein said cellulosic ether is selected from the group consisting of carboxymethylcellulose, hydroxypropyl cellulose and hydroxyethylcellulose.

134. The method of claim 130, wherein said polymeric thickening agent is selected from the group consisting of xanthan gum and guar gum.

135. The method of claim 130, wherein a concentration of said gelling agent ranges between about 0.1 weight percentage and about 5 weight percentages of the total weight of the said composition.

136. The method of claim 135, wherein a concentration of said gelling agent ranges between about 0.1 weight percentage and about 2 weight percentages of the total weight of the said composition.

137. The method of claim 105, wherein said pharmaceutical composition further comprises a penetration co-enhancer.

138. The method of claim 137, wherein said penetration co-enhancer is a glycol.

139. The method of claim 105, wherein said pharmaceutical composition further comprises an additional pharmaceutically active ingredient.

140. The method of claim 105, wherein said pharmaceutical composition further comprises at least one additive.

141. The method of claim 140, wherein said at least one additive is selected from the group consisting of a moisturizing agent and an emollient.

142. The method of claim 140, wherein a concentration of said at least one additive ranges between about 1.0 weight percentages and about 5 weight percentages of the total weight of the said composition.

143. The method of claim 14Q, wherein said at least one additive comprises glycerin.

144. The method of claim 141, wherein said emollient is selected from the group comprising dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

145. The method of claim 140, wherein said at least one additive is selected from the group consisting of a humectant, a deodorant agent, an antiperspirant, a stabilizing agent a pH adjusting agent, a preservative, an emulsifier, an occlusive agent, a solubilizing agent, a colorant, and a surfactant.

146. The method of claim 105, wherein said subject is a human male.

147. The method of claim 146, wherein said medical condition is selected from the group consisting of primary hypogonadism, secondary hypogonadism, age-related hypogonadism, hormone deficiency, erectile dysfunction, AIDS wasting syndrome, reduced sex drive, energy loss, loss of bone mass, extreme tiredness, low energy, and depression.

148. The method of claim 105, wherein said subject is a human female.

149. The method of claim 148, wherein said medical condition is selected from the group consisting of breast cancer, postpartum breast pain or engorgement, reduced sex drive, menopausal symptoms, energy loss, loss of bone mass, extreme tiredness, low energy, and depression.

150. The method of claim 148, wherein said human female is selected from the group consisting of young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

151. The method of claim 105, fisher comprising co-administering to said subject an additional pharmaceutically active ingredient suitable for treating said medical condition.
